(19) United States
(12) Patent Application Publication
(10) Pub. No.: US 2021/0290662 A1
(43) Pub. Date: Sep. 23, 2021

(54) METHODS OF TREATING UPPER GASTROINTESTINAL DISORDERS IN PPI REFRACTORY GERD

(71) Applicant: Ironwood Pharmaceuticals, Inc., Boston, MA (US)

(72) Inventors: Bernard Joseph Lavins, Lexington, MA (US); Mark G. Currie, Boston, MA (US)

(21) Appl. No.: 17/336,518

(22) Filed: Jun. 2, 2021

Related U.S. Application Data
(63) Continuation of application No. 16/424,310, filed on May 28, 2019, now abandoned.
Application No. 16/424,310 is a continuation of application No. 15/548,467, filed on Aug. 3, 2017, now abandoned.
(60) Provisional application No. 62/111,627, filed on Feb. 3, 2015.

(51) Int. Cl. A61K 31/785; A61K 45/06; A61K 47/36; A61K 47/10; A61K 9/00; A61K 47/38

(52) U.S. Cl. CPC A61K 31/785; A61K 45/06; A61K 47/36; A61K 9/0053; A61K 9/0056; A61K 47/38; A61K 47/10

(57) ABSTRACT

Disclosed herein are oral dosage forms of colesevelam, or a pharmaceutically acceptable salt thereof, adapted to treat upper gastro-intestinal disorders associated with PPI refractory GERD. Also disclosed are the methods of using these oral dosage forms to treat upper gastrointestinal disorders associated with PPI refractory, or PPI resistant, GERD in a patient in need thereof. The oral dosage forms disclosed herein are adapted for the ascribed uses by being comprised of colesevelam, or a pharmaceutically acceptable salt thereof, in a polymeric matrix comprised of one or more hydrophilic polymers such that the oral dosage form erodes upon encountering gastric fluid and has a gastric retention time of three hours or longer, allowing for an extended period of time for the colesevelam to be released in the upper GI and sequester excess bile.

### CROSS REFERENCE TO RELATED APPLICATION

This patent application is a continuation of, and claims priority under 35 U.S.C. § 120, to U.S. patent application Ser. No. 16/424,310 filed May 28, 2019, which is a continuation of U.S. patent application Ser. No. 15/548,467 filed Aug. 3, 2017, which is the United States National Phase of PCT/US2016/016044 filed Feb. 2, 2016, which claims priority under 35 U.S.C. § 119(e) to U.S. Provisional Application No. 62/111,627 filed Feb. 3, 2015, the disclosure of which applications are hereby incorporated herein by reference.

### BACKGROUND OF THE INVENTION

### Technical Field

Disclosed herein are oral dosage forms of colesevelam, or a pharmaceutically acceptable salt thereof, adapted to treat upper gastro-intestinal disorders associated with PPI refractory GERD. Disclosed herein are methods of using gastroretentive oral dosage forms of colesevelam, or a pharmaceutically acceptable salt thereof to treat upper gastrointestinal disorders associated with GERD, PPI refractory GERD, and DGER in a patient in need thereof. The oral dosage forms used in the methods of the invention are adapted for the ascribed uses by being comprised of colesevelam, or a pharmaceutically acceptable salt thereof, in a polymeric matrix comprised of one or more hydrophilic polymers such that the oral dosage form erodes upon encountering gastric fluid and has a gastric retention time of three hours or longer, allowing for an extended period of time for the colesevelam to be released in the upper GI and sequester excess bile.

### Background Information

Bile acids are steroid acids found predominantly in the bile of mammals. They are produced in the liver by the oxidation of cholesterol, and are stored in gallbladder and secreted into the intestine in the form of salts. They act as surfactants, emulsifying lipids and assisting with the absorption and digestion of dietary fat and cholesterol.

The principal bile acids are: cholic acid, chenodeoxycholic acid, deoxycholic acid, taurocholic acid, and glycocholic acid. The chemical distinctions between different bile acids are small, depending only on the presence or absence of hydroxyl groups on positions 3, 7, and 12. In humans, the most prevalent bile acids are cholic acid and chenodeoxycholic acid, and their conjugates with taurine and glycine (glycocholate and taurocholate). Some mammals synthesize predominantly deoxycholic acid. Synthesis of bile acids is a major consumer of cholesterol. The body synthesizes about 800 mg of cholesterol per day and about half of that is used for bile acid synthesis. In total, about 20-30 grams of bile acids are secreted into the intestine daily; about 90% of excreted bile acids are reabsorbed (by active transport in the ileum) and recycled. This is referred to as the enterohepatic circulation. Since bile acids are made from endogenous cholesterol, the enterohepatic circulation of bile acids may be disrupted as a way to lower cholesterol; this is the usual therapeutic rationale for administering bile acid sequestrants.

Bile acids play an important role in the digestive process; however, data from nonclinical and mechanistic studies suggest that the prolonged presence or excess of bile acids in the stomach and esophagus can result in toxic effects on regional tissues. Duodenogastroesophageal reflux (DGER), which contains bile acids, produces symptoms such as retrosternal pain, heartburn, nausea, and vomiting, and is associated with more severe esophageal pathology in patients with gastroesophagel reflux disease (GERD) and Barrett's esophagus, a pre-cancerous change in the esophagus. GERD is a chronic and common medical disorder with a prevalence estimated at approximately 20 to 40% in Western countries. GERD is associated with rising healthcare utilization and cost. Currently, PPIs are the standard of care for GERD with a standard dosing of once a day. However, approximately 10 to 40% of GERD patients remain symptomatic on this standard-dose proton pump inhibitor (PPI) therapy. This condition is commonly referred to as PPI refractory GERD or GERD that is resistant to PPI treatment.

Although commonly defined by the incomplete effectiveness of PPIs in providing relief from the effects and symptoms of GERD, the underlying causes of refractory GERD are less easily identified and are just recently being elucidated; ironically, the very effectiveness of PPIs is the primary catalyst that is allowing doctors to now explore additional or alternative mechanisms to gastric acid reflux that may contribute to the symptoms of GERD.

The primary cause of refractory GERD is now believed to be duodenogastroesophageal reflux (DGER). Patients who experience reflux of bile along with the usual acidic reflux of GERD continue to experience bothersome GERD symptoms despite treatment with PPIs.

Bile reflux can be difficult to distinguish from acid reflux because the signs and symptoms are similar, and the two conditions frequently occur at the same time. Unlike acid reflux, bile reflux inflames the stomach, often causing a gnawing or burning pain in the upper abdomen. Other signs and symptoms may include: frequent heartburn, i.e., a burning sensation in the chest that sometimes spreads to the throat along with a sour taste in the mouth; nausea; vomiting bile; a cough; or hoarseness.

Bile and stomach acid reflux into the esophagus when the lower esophageal sphincter (LES), malfunctions. The LES separates the esophagus and stomach. Normally, it opens only to allow food to pass into the stomach and then closes tightly. But if the valve relaxes abnormally or weakens, stomach acid and bile can wash back into the esophagus, causing heartburn and ongoing inflammation that may lead to serious complications.

A sticky mucous coating protects the stomach from the corrosive effects of stomach acid, but the esophagus lacks this protection, which is why bile reflux and acid reflux can seriously damage esophageal tissue. Although bile reflux can injure the esophagus on its own—even when the pH of the reflux is neutral or alkaline—the combination of bile and acid reflux seems to be particularly harmful, increasing the risk of complications.

Disorders and/or symptoms that are believed to be associated with bile reflux, either alone or in combination with acid reflux, include, for instance, heartburn, indigestion, dyspepsia, erosive esophagitis, peptic ulcer, gastric ulcer, esophageal ulcers, esophagitis, laryngitis, pharyngitis, coarse or hoarse voice, and GERD-related pulmonary dysfunction such as coughing and/or asthma. Further complications that are believed to occur as a result of chronic bile reflux are, for instance, gastroesophageal reflux disease, or GERD; Barrett's esophagus; esophageal cancer (e.g., adenocarcinoma) and gastritis.

GERD is a generic term encompassing diseases with various digestive symptoms such as pyrosis; acid regurgitation; obstructed admiration; aphagia; pectoralgia; permeating feeling (and the like) sensibility caused by reflux in the esophagus and stagnation of gastric contents, duodenal juice, pancreatic juice and the like. The term covers both reflux esophagitis, in which erosion and ulcers are endoscopically observed, and esophageal regurgitation-type non-ulcer dyspepsia (NUD) in which no abnormality is endoscopically observed. GERD occurs when the LES does not close properly and stomach contents leak back, or reflux, into the esophagus.

The inner mucosa of the esophagus is lined with non-keratinized stratified squamous epithelium arranged in longitudinal folds. Damage to the lining of the esophagus causes the normal squamous cells that line the esophagus to tum into a type of cell not usually found in humans, called specialized columnar cells. That conversion of cells in the esophagus by the acid reflux is known as Barrett's esophagus. Although people who do not have heartburn can have Barrett's esophagus, it is found about three to five times more often in people with this condition. Barrett's esophagus does not cause symptoms itself and is important only because it seems to precede the development of a particular kind of cancer esophageal adenocarcinoma. The risk of developing adenocarcinoma is 30 to 125 times higher in people who have Barrett's esophagus than in people who do not. This type of cancer is increasing rapidly in white men. This increase may be related to the rise in obesity and GERD.

Barrett's esophagus has no cure, short of surgical removal of the esophagus, which is a serious operation. Surgery is recommended only for people who have a high risk of developing cancer or who already have it. Most physicians recommend treating GERD with acid-blocking drugs, since this is sometimes associated with improvement in the extent of the Barrett's tissue. However, this approach has not been proven to reduce the risk of cancer.

Colesevelam reduces serum LDL-C levels by binding bile acids in the intestine, impeding their reabsorption. As the bile acid pool becomes depleted, the hepatic enzyme, cholesterol 7-α-hydroxylase, is upregulated, which increases the conversion of cholesterol to bile acids. This causes an increased demand for cholesterol in the liver cells, resulting in the dual effect of increasing transcription and activity of the cholesterol biosynthetic enzyme, HMG-CoA reductase, and increasing the number of hepatic LDL receptors. These compensatory effects result in increased clearance of LDL-C from the blood, resulting in decreased serum LDL-C levels. Serum triglyceride levels may increase or remain unchanged. The mechanism by which colesevelam improves glycemic control is unknown; however, increasing evidence suggests that colesevelam, as a BAS, may function by signaling molecules in the liver and GI tract for lipid and glucose metabolism. The mechanism by which colesevelam reduces symptoms of GERD and DGER is by binding bile acids that are refluxed into the stomach and preventing the free bile acids from entering the esophagus and reacting with the esophageal mucosa.

Until now, there were no reported clinical efficacy studies with colesevelam in patients with GERD or other upper GI disorders. Colesevelam has, however, been evaluated for safety in clinical studies and via post-marketing pharmacovigilance. In general, colesevelam has been shown to be safe and well-tolerated in adults with hyperlipidemia or type 2 diabetes mellitus, and in adolescents with familial hypercholesterolemia. Due to the large doses required for lipid lowering, and its local effects in the GI tract, most of the adverse events (AEs) related to colesevelam have been GI in nature (constipation, flatulence, and dyspepsia). Most of these AEs were of mild or moderate intensity. In clinical lipid-lowering trials, the incidence of dyspepsia was greater at the higher doses (3.8 and 4.5 g/day).

Protein pump inhibitors (PPIs) act by inhibiting the parietal cell H+/K+ ATPase proton pumps responsible for acid secretion from these cells. PPIs, such as omeprazole and its pharmaceutically acceptable salts, are disclosed, for example, in EP 05129, EP 124495 and U.S. Pat. No. 4,255,431. Despite their well-documented efficacy, PPIs have notable limitations. For example, patients who are non-responsive to treatment with PPI inhibitor alone may be non-responsive because even though the PPI is decreasing acid reflux from the stomach, bile acid from the duodenum is still present. Thus, an improvement of PPI-mediated activity is a well-recognized challenge in gastroenterology and there is a need in the art to address and overcome upper GI tract disorders, as well as related throat disorders as discussed above, that are non-responsive to treatment by administration of PPIs alone.

Accordingly, the development of effective methods of treating pathologies in which bile reflux is involved, either in conjunction with acid reflux or not, would be useful.

### SUMMARY OF THE INVENTION

The present invention relates to methods of using a gastroretentive oral dosage form that provides prolonged and steady levels of colesevelam in the stomach at concentrations which allow for optimal binding of bile acids refluxed from the small intestine into the stomach, thus avoiding reflux of stomach bile acids into the esophagus and other parts of the upper GI, preventing further damage.

In one embodiment, disclosed is a method of treating gastroesophageal reflux disease (GERD), refractory GERD, or duodenal-gastroesophageal reflux (DGER). The method includes administering an oral dosage form of colesevelam, or a pharmaceutically acceptable salt thereof, in a polymeric matrix comprised of one or more hydrophilic polymers to a patient in need who is already taking an acid-reducing medication.

In one embodiment, disclosed is a method of reducing the frequency or the severity of at least one symptom of gastroesophageal reflux disease (GERD), refractory GERD, or duodenal-gastroesophageal reflux (DGER). The method includes administering an oral dosage form of colesevelam, or a pharmaceutically acceptable salt thereof, in a polymeric matrix comprised of one or more hydrophilic polymers to a patient in need who is already taking an acid-reducing medication.

In one embodiment, disclosed is a method of reducing the frequency and/or the severity of a symptom in the upper gastrointestinal tract caused by bile acid reflux. The method includes administering an oral dosage form of colesevelam, or a pharmaceutically acceptable salt thereof, in a polymeric matrix comprised of one or more hydrophilic polymers to a patient in need who is already taking an acid-reducing medication.

In one embodiment, disclosed is a method of treating gastroesophageal reflux disease (GERD), refractory GERD, or duodenal-gastroesophageal reflux (DGER). The method includes administering to a patient who has one of these diseases or disorders and is currently taking a proton pump inhibitor a first dose of an oral dosage form of colesevelam, or a pharmaceutically acceptable salt thereof, in a polymeric matrix comprised of one or more hydrophilic polymers. The patient is then administered a second dose of an oral dosage form of colesevelam, or a pharmaceutically acceptable salt thereof, in a polymeric matrix comprised of one or more hydrophilic polymers. In some embodiments, each of these doses of colesevelam is between about 900 mg and about 1100 mg of colesevelam. The first dose and the second dose are given between about 4 hours and 16 hours apart.

In one embodiment, disclosed is a method of treating gastroesophageal reflux disease (GERD), refractory GERD, or duodenal-gastroesophageal reflux (DGER). The method includes administering an oral dosage form of colesevelam, or a pharmaceutically acceptable salt thereof, in a polymeric matrix comprised of one or more hydrophilic polymers to a patient in need who is already taking a proton pump inhibitor which is not entirely bound by the dosed colesevelam.

The oral dosage forms used herein are made generally by the process disclosed in WO 2014/113377, entitled Gastro-Retentive Sustained-Release Oral Dosage Form Of A Bile Acid Sequestrant. In general, this method comprises combining and blending intragranular components to form an intragranular blend. Next, the intragranular blend is compressed into slugs. These slugs are then milled to form milled granulation. Extragranular components are combined and blended to form an extragranular blend. The extragranular components and milled granulation are then combined and blended to form a dry blend. The extragranular components may be combined and blended at any time prior to their combination with the milled granulation.

More specifically, the oral dosage forms used were tablets, containing 500 mg of colesevelam hydrochloride, which were white to off-white, oval shaped and film-coated intended for oral administration. In addition to the active drug substance, colesevelam, the tablets contained the following inactive ingredients: microcrystalline cellulose, polyethylene oxide, magnesium stearate, hypromellose, and diacetylated monoglycerides.

The methods disclosed here for using the oral dosage forms require that the total amount of colesevelam, or a pharmaceutically acceptable thereof, ingested by the patient not exceed about 5000 mg a day of colesevelam. Each oral dosage form dose is between about 450 mg and about 550 mg. In some embodiments, the oral dosage form dose is about 500 mg. To be perfectly clear, for purposes of this disclosure, the term “oral dosage form” refers to the drug product; for instance, the oral dosage form may be a tablet that includes between about 450 mg and about 550 mg of colesevelam, or a tablet that includes 500 mg of colesevelam. However, in some embodiments, multiple oral dosage forms (e.g., tablets) may be given simultaneously; this may be referred to as two “doses” of the oral dosage form. For instance, two oral dosage forms (e.g., tablets) may be given together, resulting in a 900 mg to 1100 mg “dose” being given at one time. Further, the methods disclosed herein comprise dosing regimens of once daily, twice daily and thrice daily. In some embodiments, for instance, two oral dosage forms may be given in a first “dose” of 1000 mg of colesevelam in the morning, and another two oral dosage forms may be given in a second “dose” of 1000 mg of colesevelam in the evening, for a total dose of 2000 mg of colesevelam for the day.

Daily cumulative doses of less than 1000 mg may not be effective and daily cumulative doses over 5000 mg may result in the exacerbation of additional GI effect caused by excess colesevelam, including dyspepsia, nausea, bloating and constipation.

These, and other objects, features and advantages of this invention will become apparent from the following detailed description of the various aspects of the invention.

### DETAILED DESCRIPTION OF THE INVENTION

The methods disclosed herein are useful for the treatment of symptomatic GERD not completely responsive to PPIs, and other gastrointestinal (GI) disorders. Colesevelam, and its pharmaceutically acceptable salts, e.g., colesevelam hydrochloride, is a bile acid sequestrant (BAS). Colesevelam hydrochloride (hereafter referred to as colesevelam) is an orally administered, non-absorbed, non-digestible polymer that binds bile acids in the GI tract. Colesevelam has been approved by FDA since 2000 in the United States as an adjunct to diet and exercise for reduction of elevated low-density lipoprotein cholesterol in adults with primary hyperlipidemia. Colesevelam is currently available as an immediate-release formulation only. To be perfectly clear, for purposes of this application, dosages given (for instance, mg values) refer to the dose of colesevelam, even if colesevelam hydrochloride is utilized. That is, the weight of colesevelam relates to the colesevelam moiety because colesevelam HCl may require more API than colesevelam itself.

The oral dosage forms of the present invention extend the release of the colesevelam into the stomach. The released colesevelam is expected to bind bile acids that are refluxed into the stomach and upper duodenum, forming a bile acid-colesevelam complex and preventing the free bile acids from entering the esophagus. The bile acid-colesevelam complex will travel down the GI tract and be excreted without being absorbed.

The extended-release, gastric-retentive nature of the oral dosage forms is based on Depomed's Acuform® technology which utilizes swelling polymers to allow the tablet to be retained in the stomach for approximately 9 hours when dosed in the fed state, during which time the tablet slowly releases the active ingredient in the stomach. The tablet's active ingredient is steadily delivered to the stomach and upper GI tract in a near zero-order manner. The technology is used in the formulation of three FDA-approved drugs: Glumetza® (metformin HCl, extended release), Proquin® XR (ciprofloxacin HCl, extended release), and Gralise™ (gabapentin, extended release).

Colesevelam is not systemically absorbed and does not interfere with systemic drug metabolizing enzymes. Distribution of colesevelam is limited to the GI tract and elimination occurs through fecal excretion. It is possible, however, that direct interaction of certain PPIs and the oral dosage forms of the invention in the upper GI tract may diminish the activity of the PPI, perhaps by a binding mechanism. Without being held to any one theory, the mechanism may therefore be related to PPI/colesevelam binding. It might be surprisingly beneficial, therefore, to dose certain PPIs and the oral dosage forms of the invention in a manner that would minimize the possibility of such an unwanted interaction by, for example, judicious timing between the administration of the PPI and the oral dosage form of colesevelam or by the use of a PPI inhibitor that is not susceptible to the unwanted interaction.

Reference will now be made in detail to certain embodiments of the invention. While the invention will be illustrated with descriptions of specific embodiments, these descriptions are not intended to limit the invention to those embodiments. Rather, the invention is intended to cover all alternatives, modifications and equivalents that may be included within the scope of the present invention as defined by the claims. The present invention is not limited to the methods and oral dosage forms described herein but include any methods and oral dosage forms similar or equivalent to those described herein that could be used in the practice of the present invention. In the event that one or more of the incorporated literature references, patents or similar materials differ from or contradict this application, including but not limited to defined terms, term usage, described techniques or the like, this application controls with regard to the different or contradicting part and the rest of the other material is still applicable if useful in such part.

As employed above and throughout the disclosure, the following terms are provided to assist the reader. Unless otherwise defined, all terms of art, notations and other scientific or medical terms or terminology used herein are intended to have the meanings commonly understood by those of skill in the chemical, pharmaceutical and medical arts. In some cases, terms with commonly understood meanings are defined herein for clarity and/or for ready reference, and the inclusion of such definitions herein should not necessarily be construed to represent a substantial difference over the definition of the term as generally understood in the art unless otherwise indicated.

The terms “drug”, “agent”, “active pharmaceutical ingredient (API)”, “active”, “active ingredient (AI)” or “bulk active” are used indistinguishably throughout this disclosure to refer to the substance in a “pharmaceutical product” (i.e., a “medicine” or “medication” or “drug product”) that is biologically active.

As used herein, a “formulation” or “pharmaceutical composition” comprises the API and one or more pharmaceutically acceptable excipients.

The term “dosage form” or “oral dosage form”, as used herein, refers to a solid article of manufacture that has been made as described herein and in WO 2014/113377. In this particular case the oral dosage form is a gastric retentive tablet that delivers an amount of colesevelam, or its pharmaceutically acceptable salt, in the stomach over a period of time of at least 4 hours. Although certain explanations are used herein to account for the gastric retentive properties of the oral dosage forms, the exact mechanism by which this gastric retention is occurring should not limit the scope of the claims as long as the effect on gastric retention time is equivalent.

The terms “gastric fluid” and “gastric juice” are used interchangeably throughout the disclosure and refer to the endogenous fluid medium of the stomach, including water and secretions. “Simulated gastric fluid” means any fluid that is generally recognized as providing a useful substitute for authentic gastric fluid in in-vitro experiments designed to assess the chemical or biological behavior of substances in the stomach. One such simulated gastric fluid is aqueous 0.1 N HCl, pH 1.2.

The term “gastro-retentive” denotes dosage forms that provide sustained release of colesevelam as compared to conventional dosage forms or instant release forms, such as customary tablets or capsules, while avoiding an undesirably high initial dose. The release is said to be sustained because it is effected continuously over a relatively long period since the physical and chemical characteristics of the dosage form result in retention in the stomach.

A drug “release rate” as used herein, refers to the quantity of the drug released from a dosage form or pharmaceutical composition per unit time (mg/hr). Drug release rates for drug dosage forms are typically measured as an in vitro rate of dissolution, i.e., a quantity of drug released from the dosage form or pharmaceutical composition per unit time measured under appropriate conditions in a suitable fluid. Tests can be performed, for example, at about pH 1.2 (modified simulated gastric fluid, or mSGF) or at about pH 4.5 (the average pH of the stomach after a meal, simulating the fed state). Such testing may also be performed, for instance at 37° C. or 25° C. Suitable aliquots of the release rate solution (or suspension) are tested to determine the amount of drug released from the dosage form or pharmaceutical composition. A number of analytical techniques, e.g., HPLC, can be used to quantitate the amount of drug released.

As used herein, a “therapeutically or pharmaceutically effective amount” of colesevelam is an amount that, when administered to a subject with refractory GERD, will have the intended therapeutic effect, e.g., alleviation, amelioration, palliation or elimination of one or more manifestations of refractory GERD in the subject. The full therapeutic effect does not necessarily occur by administration of one dose and may occur only after administration of a series of doses. Thus, a therapeutically effective amount may be administered in one or more administrations. The term “therapeutically effective amount” as used herein also means that amount of colesevelam that elicits the biological or medicinal response in a tissue, system, animal or human that is being sought by a researcher, veterinarian, medical doctor or other clinician.

The term “pharmaceutically acceptable salt” refers to salts prepared from pharmaceutically acceptable non-toxic acids or bases including inorganic acids and bases and organic acids and bases.

Pharmaceutically acceptable salts of colesevelam may be prepared from pharmaceutically acceptable non-toxic acids including inorganic and organic acids. Suitable pharmaceutically acceptable acid addition salts acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Colesevelam hydrochloride was used in the study disclosed herein.

The preparation of the pharmaceutically acceptable salts described herein and other typical pharmaceutically acceptable salts is more fully described by Berg et al., “Pharmaceutical Salts,” J. Pharm. Sci., 1977:66:1-19, which is incorporated herein by reference.

The oral dosage forms of the invention comprise polymers, which swell upon intake of water from gastric fluid. When administered in the fed mode, when the diameter of the pyloric sphincter is contracted and reduced, these dosage forms will swell in such a manner that passage through the pyloric sphincter will be deterred such that retention times are at least 3 hours or more.

The oral dosage forms of the invention provide prolonged and steady levels of colesevelam to the stomach at concentrations which allow for optimal binding of the bile acid that has been refluxed into the stomach from the duodenum, thus avoiding bile acid damage to the stomach lining and the esophagus, and are suitable for dosing according to regimens that maximize effectives and minimizing side effects.

When contacted with the aqueous environment of use (e.g., gastric fluid), the erodible polymeric matrix imbibes water and forms an aqueous-swollen gel or matrix that entraps the bile acid sequestrant. The aqueous-swollen gel or matrix gradually erodes, swells, disintegrates and/or dissolves in the environment of use, thereby controlling the release of colesevelam in the stomach.

An essential ingredient of this water-swollen matrix is the at least one hydrophilic, water-swellable, erodible, or soluble polymer, which may generally be described as an “osmopolymer”, “hydrogel” or “water-swellable” polymer. More than one of such polymers may be combined in a dosage form of the invention in order to achieve gastric-retention as well as the desired erosion rate. The retentive properties of the dosage form may be due to thios swelling via an increase in size, change of dimensions or shape, or simply by increased weight. While the quality of being gastric retentive is an element of this invention, the manner by which the oral dosage form accomplishes this is immaterial.

The terms “hydrophilic” and “hydrophobic” are generally defined in terms of a partition coefficient P, which is defined as the ratio of the equilibrium concentration of a compound in an organic phase to that in an aqueous phase. A hydrophilic compound has a P value of less than 1.0, typically less than about 0.5, wherein P is the partition coefficient of the compound between octanol and water. A hydrophobic compound will generally have a P value greater than about 1.0, typically greater than about 5.0. The polymeric carriers herein are hydrophilic, and thus are compatible with aqueous fluids such as those present in the human body, in particular in the stomach.

The term “polymer”, as used herein, refers to a molecule containing a plurality of covalently attached monomer units, and includes branched, dendrimic and star polymers as well as linear polymers. The term includes both homopolymers and copolymers, for example random copolymers, block copolymers, and graft copolymers, as well as uncrosslinked polymers and slightly to moderately to substantially cross-linked polymers, as well as two or more inter-penetration cross-linked networks. Such polymers may be linear, branched, or cross-linked. The polymers may be homopolymers or copolymers.

The term “polyethylene oxide” or “PEO” refers to a polyethylene oxide polymer that has a wide range of molecular weights. PEO is a linear polymer of unsubstituted ethylene oxide and has a wide range of viscosity-average molecular weights. Examples of commercially available PEOs and their approximate molecular weights (in grams/mole or Daltons) are: POLYOX® NF, grade WSR coagulant, approximate molecular weight 5 million; POLYOX® grade WSR 301, approximate molecular weight 4 million; POLYOX® grade WSR 303, approximate molecular weight 7 million; POLYOX® grade WSR N60-K, approximate molecular weight 2 million; POLYOX® grade WSR N-80K, approximate molecular weight 200,000.

In one embodiment, at least one of the one or more hydrophilic polymers of the gastro-retentive oral dosage forms described herein is a swellable and erodible polymer.

In some embodiments, said polymer is a polyalkylene oxide. In some embodiments, at least one of the one or more hydrophilic polymers is a polyethylene oxide (PEO). In still other embodiments, the at least one hydrophilic polymer is a polyethylene oxide having a molecular weight of about 2,000,000 to 4,000,000 Daltons.

In other embodiments, the at least one hydrophilic polymers of the dosage form is a cellulose. In certain embodiments, the polymers may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin, corn-starch-unmodified or pregelatinized-), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

In certain embodiments, the cellulosics for the erodible matrix comprises aqueous soluble and aqueous-erodible cellulosics can include, for example, methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 Daltons, for example, the Dow Methocel™ series E5, E15LV, E50LV and KIOOLY) and high viscosity (MW greater than 50,000 Daltons, for example, E4MCR, EIOMCR, K4M, K15M and KIOOM and the Methocel™ K series) HPMC. Other commercially available types of HPMC include the Shin Etsu Metolose 90SH series.

Other materials useful as the erodible matrix material include, but are not limited to, pullulan, polyvinyl pyrrolidone (povidone), polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

The rate of drug release from the oral dosage form disclosed herein may be measured in vitro in acetate buffer at pH 4.5, using a USP Type II (paddle) apparatus with the tablets placed in sinkers. In the presently disclosed oral dosage forms, 100% drug release does not occur until after at least three hours.

### Dosage Form Preparation

The active agents used in the dosage forms of the present disclosure can be formulated in accordance with methods that are standard in the art (see e.g., Remington: the Science and Practice of Pharmacy 21st Ed. 2005, University Sciences in Philadelphia Pa.) or Developing Solid Oral Dosage Forms—Pharmaceutical Theory and Practice, 1st Ed; Academic Press; Burlington, Mass.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. Standard methods for tablet preparation include direct compression, dry granulation with roller compaction, dry granulation with slugging and wet granulation. These methods are well known to those skilled in the art.

“Granulation”, as used herein, is defined as the process in which primary powder particles are made to adhere to form larger, multi-particle entities called granules. It is the process of collecting particles together by creating bonds between them. Bonds are formed by compression (“dry granulation”) or by using a binding agent (“wet granulation”). Granulation is extensively used in the manufacturing of tablets. The granulation process generally combines one or more powder particles and forms a granule that will allow tableting process to be within required limits. This way a predictable and repeatable process is possible and quality tablets or pellets can be produced using tableting equipment.

Dry granulation can be conducted under two processes; either a large tablet (“slug”) is produced in a heavy duty tableting press (“slugging”) or the powder is squeezed between two rollers to produce ribbons of materials (“roller compaction”). These materials (i.e., the slugs or the ribbons) are then milled to provide the “granules”.

In accordance with the invention, when the ingredients are incorporated prior to granulation, they are referred to as “intragranular”, i.e., within the granule. When the ingredients are incorporated after granulation, they are referred to as “extragranular”.

The enteric coating surrounding the core may be applied using standard coating techniques. Materials used to form the enteric coating may be dissolved or dispersed in organic or aqueous solvents and may include one or more of the following: methacrylic acid copolymers; shellac; hydroxypropylmethylcellulose phthalate; polyvinyl acetate phthalate; hydroxypropylmethylcellulose trimellitate; carboxymethylcellulose; cellulose acetate phthalate; or other suitable enteric coating polymers. The pH at which the enteric coat will dissolve can be controlled by the polymer or combination of polymers selected and/or ratio of pendant groups. For example, dissolution characteristics of the coating can be altered by the ratio of free carboxyl groups to ester groups.

In some embodiments, the coating of the unit dosage form of the invention comprises a microcrystalline cellulose and an acetylated glyceride.

In some embodiments, the gastro-retentive sustained release dosage forms of the invention can be prepared by a process as described below. Intragranular components are combined and blended to form an intragranular blend. In some instances, the bile acid sequestrant (active ingredient) is one of the intragranular components. The intragranular components may further include fillers or compression aids, such as microcrystalline cellulose, and/or lubricants, such as magnesium stearate. The intragranular blend is compressed into slugs, and the slugs are milled to form milled granulation. The yield for the milled granulation is calculated so that the desired amounts of the extragranular components to be used can be determined. Extragranular components are combined and blended to form an extragranular blend. In some instances, the hydrophilic polymer is one of the extragranular components. There may be more than one hydrophilic polymer present. In some instances, the hydrophilic polymer may be comprised of polyalkylene oxide, such as polyethylene oxide. The extragranular components may include fillers or compression aids, such as microcrystalline cellulose; binders or drug release aids, such as trehalose or hydroxypropylmethylcellulose; plasticizers, such as diacetylated monoglyceride; and/or lubricants, such as magnesium stearate. The extragranular components and milled granulation are then combined and blended to form a dry blend. The extragranular components may be combined and blended at any time prior to their combination with the milled granulation; that is the extragranular components may be combined and blended before the intragranular components are combined and blended, or vice-versa.

In some embodiments, the dry blend may be compressed into one or more tablets. In other embodiments, the tablets may be coated with an outer layer (coating). In some embodiments, the coating may be 3:1 HPMC (Grade-E50 Premium LV): diacetylated monoglycerides, NF Grade-(Myvacet 9-45K).

The dosage forms of the invention may be packaged for use in a variety of ways depending upon the method used for administering the drug. Generally, an article for distribution includes a container having deposited therein the pharmaceutical dosage form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

### Therapeutic Methods

In one aspect, disclosed is a method of treating gastroesophageal reflux disease (GERD), refractory GERD, or duodenal-gastroesophageal reflux (DGER). The method includes administering an oral dosage form of colesevelam, or a pharmaceutically acceptable salt thereof, in a polymeric matrix comprised of one or more hydrophilic polymers to a patient in need who is already taking an acid-reducing medication. In some embodiments, the gastro retentive oral dosage form comprise a dose of colesevelam between 450 mg and 550 mg. In some embodiments, the oral dosage form dose is 500 mg. In some embodiments, the oral dosage form may be administered in one dose. In some embodiments, the oral dosage form may be administered as two doses. In some embodiments, the oral dosage form may be administered as three doses. In some embodiments, multiple oral dosage forms may be given at one time. In some embodiments, two oral dosage forms may be given at one time. In some embodiments, three oral dosage forms may be given at one time. For instance, two doses of the oral dosage form may be given together, resulting in 900 mg to 1100 mg being given at one time.

In some embodiments, the oral dosage form is administered one time in a 24-hour period. In some embodiments, the oral dosage form is administered two times in a 24-hour period. In some embodiments, the oral dosage form is administered three times in a 24-hour period.

In some embodiments, the total daily dose of colesevelam administered is between about 450 mg and about 5000 mg. In some embodiments, the total daily dose of colesevelam administered is between about 450 mg and about 1850 mg. In some embodiments, the total daily dose of colsevelam administered is between about 1350 mg and about 4950 mg. In some embodiments, the total daily dose of colesevelam administered is between about 900 mg and about 3300 mg. In some embodiments, the total daily dose of colesevelam administered is between about 1800 mg and about 2200 mg. In some embodiments, the total daily dose of colesevelam administered is 2000 mg.

In some embodiments, a first dose of the oral dosage form of colesevelam is administered between about 4 hours and about 16 hours before the administration of a second dose of the oral dosage form of colesevelam. In some embodiments, a first dose of the oral dosage form of colesevelam is administered between about 8 hours and about 12 hours before the administration of a second dose of the oral dosage form of colesevelam. In some embodiments, a first dose of the oral dosage form of colesevelam is administered between about 4 hours and about 8 hours before the administration of a second dose of the oral dosage form of colesevelam. In some embodiments, a first dose of the oral dosage form of colesevelam is administered between about 8 hours and about 16 hours before the administration of a second dose of the oral dosage form of colesevelam. In some embodiments, a first dose of the oral dosage form of colesevelam is administered between about 6 hours and about 12 hours before the administration of a second dose of the oral dosage form of colesevelam. In some embodiments, a first dose of the oral dosage form of colesevelam is administered between about 6 hours and about 10 hours before the administration of a second dose of the oral dosage form of colesevelam. In some embodiments, a first dose of the oral dosage form of colesevelam is administered between about 8 hours and about 10 hours before the administration of a second dose of the oral dosage form of colesevelam. In some embodiments, a first dose of the oral dosage form of colesevelam is administered between about 6 hours and about 14 hours before the administration of a second dose of the oral dosage form of colesevelam.

In some embodiments, at least one dose of the oral dosage form of colesevelam described herein is administered with one or more meals. In some embodiments, the dose of oral dosage form of colesevelam described herein is administered at bedtime. In some embodiments, the dose of oral dosage form of colesevelam described herein is administered with one or more meals and at bedtime. In some embodiments, the dose of oral dosage form of colesevelam described herein is administered before or after one or more meals. In some embodiments, the dose of oral dosage form of colesevelam described herein is administered with a meal. In some embodiments, the dose of oral dosage form of colesevelam described herein is administered up to 30 minutes after a meal. In some embodiments, the dose of oral dosage form of colesevelam described herein is administered up to 5 minutes before a meal.

In some embodiments, the acid-reducing medication is administered between about 0.5 hours and about 4 hours before the administration of a first dose of colesevelam. In some embodiments, the acid-reducing medication is administered between about 1 hour and about 2 hours before the administration of a first dose of colesevelam. In some embodiments, the acid-reducing medication is administered between about 0.5 hours and about 2 hours before the administration of a first dose of colesevelam. In some embodiments, the acid-reducing medication is administered between about 0.5 hours and about 1 hour before the administration of a first dose of colesevelam. In some embodiments, the acid-reducing medication is administered between about 1.5 hours and about 2 hours before the administration of a first dose of colesevelam. In some embodiments, the acid-reducing medication is administered between about 0.5 hours and about 3 hours before the administration of a first dose of colesevelam. In some embodiments, the acid-reducing medication is administered between about 2 hours and about 4 hours before the administration of a first dose of colesevelam. In some embodiments, the acid-reducing medication and the first dose of the oral dosage form of colesevelam can be administered simultaneously (also referred to as “conjointly” or “comcomitantly”).

In some embodiments, disclosed is a method of reducing the frequency or the severity of at least one symptom of gastroesophageal reflux disease (GERD), refractory GERD, or duodenal-gastroesophageal reflux (DGER). The method includes administering an oral dosage form of colesevelam, or a pharmaceutically acceptable salt thereof, in a polymeric matrix comprised of one or more hydrophilic polymers to a patient in need who is already taking an acid-reducing medication. In some embodiments, the symptom is selected from the group consisting of pain, epigastric burning, early fullness, post-prandial fullness, regurgitation, and heartburn.

The terms, “disease”, “disorder” and “condition” may be used interchangeably here to refer to a medical or pathological condition or symptom that is believed to be the result of bile reflux.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human.

A “susceptible individual” or “a patient in need thereof” is an individual who suffers from, is suffering from, or is likely to or predisposed to suffer from an upper GI tract or a throat disorder that is believed to be result of bile reflux. In humans, and as used herein, these conditions may include, for example heartburn, indigestion, dyspepsia, erosive esophagitis, peptic ulcer, gastric ulcer, esophageal ulcers, esophagitis, laryngitis, pharyngitis, coarse voice, and GERD-related pulmonary dysfunction such as coughing and/or asthma.

Further complications that are believed to occur as a result of bile reflux are, for instance, gastroesophageal reflux disease, or GERD; Barrett's esophagus; esophageal cancer (e.g., adenocarcinoma) and gastritis. In animals these conditions may include, for example, peptic ulcer of the forestomach.

The term “biological sample”, as used herein, refers to an in vitro or ex vivo sample, and includes, without limitation, cell cultures or extracts thereof; biopsied material obtained from a mammal or extracts thereof; blood, saliva, urine, faeces, semen, tears, lymphatic fluid, ocular fluid, vitreous humour, or other body fluids or extracts thereof.

“Treat”, “treating” or “treatment” with regard to a disorder or disease refers to alleviating or abrogating the cause and/or the effects of the disorder or disease. Treatment can involve administering a compound described herein to a patient diagnosed with a disease, and may involve administering the compound to a patient who does not have active symptoms. Conversely, treatment may involve administering the compositions to a patient at risk of developing a particular disease, or to a patient reporting one or more of the physiological symptoms of a disease, even though a diagnosis of this disease may not have been made.

As used herein, “treating” or “treatment of” a condition or subject refers to taking steps to obtain beneficial or desired results, including clinical results. For purposes of this disclosure, beneficial or desired clinical results include, but are not limited to, alleviation or amelioration of one or more disease, symptom, or condition that arises as a result of bile reflux.

The terms “administer”, “administering” or “administration” in reference to a dosage form of the invention refers to the act of introducing the dosage form into the system of subject in need of treatment. When a dosage form of the invention is given in combination with one or more other active agents (in their respective dosage forms), “administration” and its variants are each understood to include concurrent and/or sequential introduction of the dosage form and the other active agents.

Administration of any of the described dosage forms includes parallel administration, co-administration or sequential administration, in which the therapies are administered at approximately the same time, e.g., within about a few seconds to a few hours of one another.

The term “fed mode”, as used herein, refers to a state which is typically induced in a patient by the presence of food in the stomach, the food giving rise to two signals, one that is said to stem from stomach distension and the other a chemical signal based on food contents in the stomach. It has been determined that once the fed state is induced, larger particles are retained in the stomach for a longer period of time than smaller particles. The fed mode is induced by nutritive materials entering the stomach upon the ingestion of food. Initiation of the fed state is accompanied by a rapid and profound change in the motor pattern of the upper GI tract, over a period of 30 seconds to one minute. The change is observed almost simultaneously at all sites along the GI tract and occurs before the stomach contents have reached the distal small intestine. Once the fed state is established, the stomach generates 3-4 continuous and regular contractions per minute, similar to those in the fasted mode but with about a quarter to half the amplitude (Force). The pylorus is partially opened, causing a sieving effect in which liquids and small particles flow continuously from the stomach into the intestine while indigestible particles greater in size than the pyloric opening are retropelled and retained in the stomach. This effect causes the stomach to retain particles exceeding about 1 cm in size for approximately 4 to 8 hours or more.

Administration of a dosage form “with a meal”, as used herein, refers to administration during or after the ingestion of food or drink. When the dosage form is administered after a meal, it may be administered about 1, 2, 3, 4, 5, 10, 15 or up to 30 minutes after completion of a meal. In some embodiments, the dosage form may be administered up to 30 minutes after a meal. In some embodiments, the dosage form may be administered up to 5 minutes before a meal.

In another aspect, the patient has a genetic predisposition to developing a bile reflux related disorder. In another aspect, a dosage form herein described, is administered to a patient in order to prevent or minimize damage to the upper GI tract.

In one embodiment, the methods of the invention are a preventative or “preemptive” measure to a patient, specifically a human, having a predisposition (e.g. a genetic predisposition) to developing a disease, disorder or symptom believed to be the result of bile reflux.

In other embodiments, the methods of the invention are a preventative or “preemptive” measure to a patient, specifically a human, suffering from a disease, disorder or condition that makes him at risk of developing a bile reflux related disorder or symptom.

The gastric-retentive sustained-release oral dosage forms here disclosed are also useful for veterinary treatment of companion animals, exotic animals and farm animals, including, without limitation, dogs, cats, mice, rats, hamsters, gerbils, guinea pigs, rabbits, horses, pigs and cattle.

The term “acid-reducing medication,” as used herein, includes any medication that reduces or neutralizes stomach acids. In some embodiment, the acid-reducing medication is a proton pump inhibitor. In other embodiments, the acid-reducing medication is a histamine H₂-receptor antagonist. In some embodiments, the acid-reducing medication is an antacid. In some embodiments, the acid-reducing medication is L-arginine. In some embodiments, the acid-reducing medication is glycine.

In some embodiments, the oral dosage form is administered at a dose of 1000 mg of colesevelam, two times per day, wherein the total daily dose of colesevelam is 2000 mg.

PPI drugs are substituted benzimidazole compounds that specifically inhibit gastric acid secretion by affecting the H+/K+ ATPase enzyme system (the proton pump). These drugs, for example esomeprazole, are rapidly absorbed and have very short half-lives. However, they exhibit prolonged binding to the H+/K+ ATPase enzyme. The anti-secretory effect reaches a maximum in about 4 days with once-daily dosing. Because of these characteristics, patients beginning PPI therapy do not receive maximum benefit of the drug and healing may not begin for up to 5 days after therapy begins when PPis are used alone for initial therapy of upper GI tract disorders.

Proton pump inhibitors (PPis) are potent inhibitors of gastric acid secretion, inhibiting H+/K+ ATPase, the enzyme involved in the final step of hydrogen ion production in the parietal cells. The term proton pump inhibitor includes, but is not limited to, omeprazole (as sold under the brand-names PRILOSEC®, LOSEC®, or ZEGERID®), lansoprazole (as sold under the brand-name PREYACID®, ZOTON®, or INHIBITOL®), rabeprazole (as sold under the brand-name RABECID®, ACIPHEX®, or PARIET®), pantoprazole (as sold under the brand-name PROTONIX®, PROTIUM®, SOMAC®, or PANTOLOC®), tenatoprazole (also referred to as benatoprazole), and leminoprazole, including isomers, enantiomers and tautomers thereof (e.g., esomeprazole (as sold under the brand-name NEXIUM®)), Dexlansoprazole, Dexrabeprazole, (S)-Pantoprazole, Ilaprazole and alkaline salts thereof. The following patents describe various benzimidazole compounds suitable for use in the disclosure described herein: U.S. Pat. Nos. 4,045,563, 4,255,431, 4,359,465, 4,472,409, 4,508,905, JP-A-59181277, U.S. Pat. Nos. 4,628,098, 4,738,975, 5,045,321, 4,786,505, 4,853,230, 5,045,552, EP-A-295603, U.S. Pat. No. 5,312,824, EP-A-166287, U.S. Pat. No. 5,877,192, EP-A-519365, EP5129, EP 174,726, EP 166,287 and GB 2,163,747. All of the above patents are hereby incorporated herein by reference. Thus, proton pump inhibitors and their pharmaceutically acceptable salts, which are used in accordance with the present disclosure, are known compounds and can be produced by known processes. In certain embodiments, the proton pump inhibitor is omeprazole, either in racemic mixture or only the (−)enantiomer of omeprazole (i.e. esomeprazole), as set forth in U.S. Pat. No. 5,877,192, hereby incorporated by reference.

Omeprazole is typically administered in a 20 mg dose/day for active duodenal ulcer for 4-8 weeks; in a 20 mg dose/day for gastro-esophageal reflux disease (GERD) or severe erosive esophagitis for 4-8 weeks; in a 20 mg dose/twice a day for treatment of Helicobacter pylori (in combination with other agents); in a 60 mg dose/day for active duodenal ulcer for 4-8 weeks and up to 120 mg three times/day, and in a 40 mg dose/day for gastric ulcer for 4-8 weeks. Such dosages are contemplated to be within the scope of the present disclosure. Thus, in certain embodiments of the present disclosure, the amount of proton pump inhibitor which is included in the dosage form is an amount which is considered to be therapeutically effective, in accordance with the dosages set forth above for a variety of disease states. In other embodiments of the present disclosure, the dose of proton pump inhibitor is sub therapeutic. For example, when the drug is omeprazole, the dosage form may contain from about 0.1 mg to about 120 mg omeprazole.

Lansoprazole is typically administered about 15-30 mg/day; rabeprazole is typically administered 20 mg/day and pantoprazole is typically administered 40 mg/day. However, any therapeutic or sub-therapeutic dose of these agents is considered within the scope of the present disclosure.

H₂ blockers are drugs that inhibit the production of acid in the stomach. Exemplary histamine H₂-receptor antagonists include, for example, cimetidine (as sold under the brand-name TAGAMET HB®), famotidine (as sold under the brand-name PEPCID AC®), nizatidine (as sold under the brand-name AXID AR®), and ranitidine (as sold under the brand-name ZANTAC 75®).

Antacids work by chemical neutralization of the acid, such as sodium bicarbonate, or by absorption of the acid, such as calcium and magnesium salts. Antacids generally have a rapid onset of action and short duration. Most antacids are the conjugate bases of mild acids. Examples of common antacids include, but are not limited to, Alka-Seltzer, NaHCO₃ and/or KHCO₃; Brioschi, CHNaO₃; Gaviscon, Al(OH)₃; Maalox (liquid), Al(OH)₃ and Mg(OH)₂; Maalox (tablet), CaCO₃; Milk of Magnesia, Mg(OH)₂; Pepto-Bismol, C₇H₅BiO₄; Pepto-Bismol Children's, CaCO₃; Rolaids, CaCO₃ and Mg(OH)₂; and Tums, CaCO₃.

### Kits

Kits for treating an upper GI tract or throat disorder comprising, in one or more containers, a an oral dosage form of colesevelam, or a pharmaceutically acceptable salt thereof, in a polymeric matrix comprised of one or more hydrophilic polymers described herein, and a label or packaging insert containing instructions for use are disclosed.

The compounds and pharmaceutical formulations described herein may be contained in a kit. The kit may include single or multiple doses of two one or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a dosage form described herein (one or more than one units to make up the necessary therapeutic dosage); and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for co therapy use, a pharmaceutically acceptable carrier, vehicle or diluent.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide written memory aid containing information and/or instructions for the physician, pharmacist or subject regarding when the medication is to be taken. A “daily dose” can be a single tablet or several tablets to be taken on a given day. When the kit contains separate compositions, a daily dose of one or more compositions of the kit can consist of one tablet or capsule while a daily dose of another or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

These and other objects, features and advantages of this disclosure will become apparent from the following detailed description of the various aspects of the disclosure taken in conjunction with the accompanying Examples.

### EXAMPLES

All references provided in the Examples are herein incorporated by reference in their entirety. As used herein, all abbreviations, symbols and conventions are consistent with those used in the contemporary scientific literature. See, e.g. Janet S. Dodd, ed., The ACS Style Guide: A Manual for Authors and Editors, 2^{nd} Ed., Washington, D.C.: American Chemical Society, 1997, herein incorporated in its entirety by reference.

### Clinical Studies

A clinical study was conducted to evaluate the effect of a gastroretentive oral dosage form of colesevelam hydrochloride administered for 4 weeks in patients with GERD that was not completely responsive to proton pump inhibitors. The objective of the study was to assess the effect of a gastroretentive oral dosage form of colesevelam dosed twice daily (BID) as an adjunct treatment to a once daily (QD) proton pump inhibitor (PPI) on duodenogastroesophageal reflux (DGER) and associated symptoms in patients with gastroesophageal reflux disease (GERD) who were not completely responsive to QD PPI therapy. Each gastroretentive oral unit dosage contained 500 mg of colesevelam hydrochloride.

The study was a multicenter, randomized, double-blind, placebo-controlled, parallel-group, 4-week study. The study enrolled patients with GERD that was not completely responsive to QD PPI therapy. The patients were then randomized in a 1:1 ratio into one of two treatment groups (approximately 45 patients per group): twice daily (BID) administration of a gastroretentive oral dosage form containing 2×500 mg of colesevelam hydrochloride (2000 mg total daily dose) or placebo. In addition, randomization was stratified by baseline bile reflux status.

The trial included a two-week pretreatment period during which baseline symptoms were assessed via an electronic diary, followed by a randomization period in which patients had the option to undergo 24-hour Bilitec® and pH monitoring to assess the extent of esophageal exposure to bile and acid reflux. Patients were randomized to receive either gastric retentive dosage forms containing colesevelam or placebo for four weeks. Patients continued to take their PPI during the pretreatment, randomization and treatment periods. The exploratory study evaluated a number of GERD-related symptoms rather than specifying a primary endpoint, and as such was not powered to establish the statistical significance of a particular endpoint. Data presented for daytime heartburn severity and heartburn-free days reflect change from baseline to week four. For the responder analysis, responders regarding degree of relief of overall GERD symptoms were defined as patients who reported scores of 1 (completely relieved) or 2 (considerably relieved) on a seven-point scale for at least two out of four weeks in the treatment period, or who reported scores of 1, 2 or 3 (somewhat relieved) for all four weeks.

The initial data from the study confirmed the hypothesis that some refractory GERD patients experience bile reflux into the esophagus. Approximately two-thirds, or 33 of the 52 patients who underwent bile reflux monitoring, tested positive for bile reflux into the esophagus during the pretreatment period of the study. Importantly, the subgroup of patients in this study who tested positive for bile reflux and received active dosage forms demonstrated encouraging improvements in relief of heartburn and certain other upper gastrointestinal symptoms often associated with refractory GERD, when compared to patients receiving placebo.

Heartburn was the most severe and most frequent symptom experienced by patients before starting study treatment. Average baseline heartburn severity among study participants was 3.4 on a 10-point scale, with 0 representing no heartburn and 10 representing very severe heartburn. The improvement in daytime heartburn severity for patients treated with active dosage forms was 1.7 points in the overall trial population and 2.1 points in the subgroup of patients who tested positive for bile reflux (versus 1.2 points and 1.1 points, respectively, for the placebo-treated groups in each comparison). In terms of frequency, patients entering the trial reported that only 13.7% of their days were free of heartburn. The percentage of heartburn-free days for patients treated with active dosage forms increased by 30.3% in the overall trial population and 34.6% in the bile reflux-positive subgroup (versus 24.7% and 23.6%, respectively, for the placebo-treated groups in each analysis).

Patients receiving active dosage forms also demonstrated encouraging improvements in regurgitation and in some upper GI symptoms that are often associated with GERD, including epigastric burning, early fullness and post-prandial fullness. Symptom improvements were greatest in the bile reflux-positive subgroup. Additionally, 45.7% and 56.3% of patients treated with active dosage form in the overall trial population and in the bile reflux-positive subgroup, respectively, were responders regarding degree of relief of overall GERD symptoms (versus 27.7% and 29.4%, respectively, for the placebo-treated groups in each analysis). Certain upper GI symptoms that did not appear to be impacted by treatment included nausea, epigastric pain and bloating. The gastric retentive dosage forms of colesevelam were generally well-tolerated with the most common adverse event being constipation.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A method of treating a disease or disorder selected from gastroesophageal reflux disease (GERD), refractory GERD, or duodenal-gastroesophageal reflux (DGER), comprising administering to a patient in need thereof an oral dosage form of colesevelam, or a pharmaceutically acceptable salt thereof, in a polymeric matrix comprised of one or more hydrophilic polymers, wherein said patient in need thereof is on concomitant therapy with an acid-reducing medication.

2. A method of reducing the frequency or the severity of at least one symptom of gastroesophageal reflux disease (GERD), refractory GERD, or duodenal-gastroesophageal reflux (DGER), comprising administering to a patient in need thereof an oral dosage form of colesevelam, or a pharmaceutically acceptable salt thereof, in a polymeric matrix comprised of one or more hydrophilic polymers, wherein said patient in need thereof is on concomitant therapy with an acid-reducing medication.

3. A method of reducing the frequency or the severity of at least one symptom in the upper gastrointestinal tract caused by bile acid reflux, comprising administering to a patient in need thereof an oral dosage form of colesevelam, or a pharmaceutically acceptable salt thereof, in a polymeric matrix comprised of one or more hydrophilic polymers, wherein said patient in need thereof is on concomitant therapy with an acid-reducing medication.

4. The method according to any one of claims 1-3, wherein said oral dosage form comprises between about 450 mg and about 550 mg of colesevelam.

5. The method according to claim 4, wherein said oral dosage form comprises about 500 mg of colesevelam.

6. The method according to claim 4, wherein said oral dosage form is administered in one dose, two doses or three doses.

7. The method according to claim 6, wherein one dose of said oral dosage form is administered at one time.

8. The method according to claim 6, wherein two doses of said oral dosage form are administered at one time.

9. The method according to claim 6, wherein three doses of said oral dosage form are administered at one time.

10. The method according to any one of claims 1-9, wherein said oral dosage form is administered one time, two times, or three times in a 24-hour period.

11. The method according to claim 10, wherein the oral dosage form is administered one time in a 24-hour period.

12. The method according to claim 10, wherein the oral dosage form is administered two times in a 24-hour period.

13. The method according to claim 10, wherein the oral dosage form is administered three times in a 24-hour period.

14. The method according to any one of claims 1-3, wherein the total daily dose of colesevelam administered is between about 450 mg and about 5000 mg.

15. The method according to claim 14, wherein the total daily dose of colesevelam administered is between about 450 mg and about 1850 mg.

16. The method according to claim 14, wherein the total daily dose of colesevelam administered is between about 1350 mg and about 4950 mg.

17. The method according to claim 14, wherein the total daily dose of colesevelam administered is between about 900 mg and about 3300 mg.

18. The method according to claim 17, wherein the total daily dose of colesevelam administered is between about 1800 mg and about 2200 mg.

19. The method according to claim 18, wherein the total daily dose of colesevelam administered is 2000 mg.

20. The method according to any one of claims 1-3, wherein a first dose of colesevelam is administered between about 4 hours and about 16 hours before the administration of a second dose of colesevelam.

21. The method according to claim 20, wherein a first dose of colesevelam is administered between about 8 hours and about 12 hours before the administration of a second dose of colesevelam.

22. The method according to any one of claims 1-3, wherein said acid-reducing medication is selected from the group consisting of a proton pump inhibitor, an H2 receptor blocker, an antacid, L-arginine, and glycine.

23. The method according to claim 22, wherein said acid-reducing medication is a proton pump inhibitor.

24. The method according to claim 23, wherein said proton pump inhibitor is selected from the group consisting of omeprazole, esomeprazole, lansoprazole, pantoprazole, rabeprazole, tenatoprazole, leminoprazole, dontoprazole, and ransoprazole.

25. The method according to claim 20, wherein at least one dose of colesevelam is administered with a meal.

26. The method according to claim 20, wherein at least one dose of colesevelam is administered up to 30 min after a meal.

27. The method according to claim 20, wherein at least one dose of colesevelam is administered up to 5 minutes before a meal.

28. The method according to any one of claims 1-3, wherein the oral dosage form is administered at a dose of 1000 mg of colesevelam, two times per day, wherein the total daily dose of colesevelam is 2000 mg.

29. The method according to any one of claims 1-3, wherein said acid-reducing medication is administered between about 0.5 hours and about 4 hours before the administration of a first dose of colesevelam.

30. The method according to claim 29, wherein said acid-reducing medication is administered between about 1 hour and about 2 hours before the administration of a first dose of colesevelam.

31. The method according to claim 29, wherein said acid-reducing medication is administered between about 0.5 hours and about 2 hours before the administration of a first dose of colesevelam.

32. The method according to claim 31, wherein said acid-reducing medication is administered between about 0.5 hours and about 1 hour before the administration of a first dose of colesevelam.

33. The method according to claim 29, wherein said acid-reducing medication is administered between about 1.5 hours and about 2 hours before the administration of a first dose of colesevelam.

34. The method according to claim 29, wherein said acid-reducing medication is administered between about 0.5 hours and about 3 hours before the administration of a first dose of colesevelam.

35. The method according to claim 29, wherein said acid-reducing medication is administered between about 2 hours and about 4 hours before the administration of a first dose of colesevelam.

36. The method according to any one of claims 1-3, wherein said acid-reducing medication and the first dose of colesevelam are administered simultaneously.

37. A method according to claim 1 or claim 2, wherein said disease is refractory gastroesophageal reflux disease.

38. The method according to claim 2 or claim 3, wherein said symptom is selected from the group consisting of pain, epigastric burning, early fullness, post-prandial fullness, regurgitation, and heartburn.

39. A method of treating gastroesophageal reflux disease (GERD), refractory GERD, or duodenal-gastroesophageal reflux (DGER), comprising administering to a patient in need thereof a first dose of an oral dosage form of colesevelam, or a pharmaceutically acceptable salt thereof, in a polymeric matrix comprised of one or more hydrophilic polymers; and a second dose of an oral dosage form of colesevelam, or a pharmaceutically acceptable salt thereof, in a polymeric matrix comprised of one or more hydrophilic polymers; wherein each of said first and second doses of colesevelam comprises between about 900 mg and about 1100 mg of colesevelam; wherein said first gastroretentive oral dosage form and said second gastroretentive oral dosage form are given between about 4 hours and 16 hours apart; and wherein said patient in need thereof is on concomitant therapy with a proton pump inhibitor.

40. A method of treating a disease or disorder selected from gastroesophageal reflux disease (GERD), refractory GERD, or duodenal-gastroesophageal reflux (DGER), comprising administering to a patient in need thereof an oral dosage form of colesevelam, or a pharmaceutically acceptable salt thereof, in a polymeric matrix comprised of one or more hydrophilic polymers, wherein said patient in need thereof is on concomitant therapy with a proton pump inhibitor, and wherein said proton pump inhibitor is not entirely bound by said colesevelam.
